(19) United States
(12) Patent Application Publication
(10) Pub. No.: US 2010/0221823 A1
(43) Pub. Date: Sep. 2, 2010

(54) METHOD FOR CULTURING MAMMALIAN CELLS TO IMPROVE RECOMBINANT PROTEIN PRODUCTION

(75) Inventors: Rebecca E. McCoy, Port Orchard, WA (US); Arvia E. Morris, Seattle, WA (US)

Correspondence Address: AMGEN INC.;LAW DEPARTMENT, 1201 AMGEN COURT WEST, SEATTLE, WA, 98119, US

(73) Assignee: Amgen Inc., Thousand Oaks, CA (US)

(21) Appl. No.: 12/452,000

(22) PCT Filed: Jun. 10, 2008
(86) PCT No.: PCT/US2008/007254

§ 371 Date: Apr. 16, 2010

Related U.S. Application Data
(60) Provisional application No. 60/943,212, filed on Jun. 11, 2007.

(51) Int. Cl. C12N 5/10; C12N 5/071

(52) U.S. Cl. 435/328; 435/358; 435/326

(57) ABSTRACT

The present invention relates to methods for mammalian cell culture, wherein the methods make use of media containing polyamines, such as putrescine, spermidine and spermine.

### CROSS-REFERENCE TO RELATED APPLICATIONS

This application claims the benefit of U.S. provisional application Ser. No. 60/943,212, filed Jun. 11, 2007, the disclosure of which is relied upon and incorporated by reference herein.

### FIELD OF INVENTION

The present invention relates to methods for mammalian cell culture, wherein the methods make use of media containing polyamines, such as putrescine, spermidine, and spermine.

### BACKGROUND OF INVENTION

Many commercially important proteins are produced in mammalian cell lines that are adapted for long term growth in culture. Chinese hamster ovary (CHO) cell lines are well suited and widely used for recombinant production of therapeutic proteins, also known as “biologics”, including recombinant antibodies. CHO cell lines efficiently produce proteins that are correctly folded and have desired post-translational modifications. Further, CHO cell lines have gained acceptance and approval by regulatory agencies for use in clinical manufacturing of recombinant protein therapeutics.

To facilitate recombinant production of proteins, new culture media are being developed and/or known media are being improved to meet the nutritional requirements of host cell lines and production parameters, especially for large scale culture processes. Cell culture media formulations are described in the literature and a growing number of media are commercially available. Many formulations rely upon animal sera, such as fetal bovine serum (FBS), to facilitate cell growth, viability and protein production. However, use of serum is problematic for large scale clinical manufacture of protein therapeutics. Not only is large scale use of serum prohibitively expensive, serum is inherently uncharacterized, comprised of complex, unknown and unquantified components. The quality and composition of serum is highly variable among different animal sources and between different manufacturers, even varying between lots from a single manufacturer. This inherent variability makes predictable large scale cell culture and/or recombinant protein production difficult and expensive. In addition, removing serum proteins from downstream processing is burdensome. Even without these difficulties, use of serum in a clinical manufacturing setting is highly undesirable from a regulatory point of view because animal serum brings with it the risk of contamination by viruses, mycoplasma and/or prions.

Simply removing serum from a cell culture media formulation is not the answer. The components that give serum its complexity and variability also contribute to robust cell growth, viability and protein production. One method is to replace animal sera with animal and/or plant protein hydrolysates or “peptone”. Addition of peptone of serum-free cell culture media can stimulate vigorous cell growth, viability and protein production. However, these hydrosylates are essentially undefined and, like animal sera, contain many complex, unknown and unquantified components. In order to maintain the benefits of sera and peptone without the detrimental aspects, these undefined components must be identified and added back to serum-free, peptone-free or “defined media”. Much effort has been given to identifying these components and their optimum concentration ranges, in an effort to develop serum-free, peptone-free media and/or cell culture formulations where each of the components is defined, the media performs as well or exceeds that of a sera or peptone supplemented media. These defined cell culture media formulations are better suited to large scale recombinant protein production processes typical in clinical manufacturing. Defined media formulations allow greater flexibility for optimization and improvements to cell growth and recombinant protein production including increasing cell growth rates, growth to high cell densities, controlling the stage and amount of cell differentiation, increasing protein secretion, increasing phenotypic and genetic stability and elimination of senescence for many cell types.

Clinical manufacture of therapeutic proteins is an expensive, large scale endeavor. Maintaining cell growth and viability through out the cell culture process is very important, increased recombinant protein production at the expense of diminished cell viability and/or growth can be counterproductive. Positive increases in protein production, cell growth and viability are beneficial to the production of protein therapeutics. New cell culture media components, formulations and/or optimization of components that provide even incremental improvements in cell growth, viability and/or protein production are valuable, given the expense of large scale cell culture processes and the difficulty and expense of building and obtaining regulatory approval for new large-scale, commercial culture facilities.

There is a continuing need to develop cell culture media which optimizes cell growth and viability and increases recombinant protein production. Any improvements to recombinant polypeptide expression, titer, cell growth and/or cell viability can lead to higher production levels, thereby reducing costs associated with the manufacture of protein therapeutics. The invention fulfills these needs by providing simple, easy and inexpensive methods of increasing cell growth and protein production.

### SUMMARY OF THE INVENTION

The present invention provides a method comprising culturing an animal cell line expressing a protein of interest in serum free cell culture medium; the medium comprising spermine or spermidine at a concentration of at least about 0.10 μM, or putrescine at a concentration of at least about 100 μM. The medium may also comprise combinations of spermine, spermidine and putrescine at these concentrations. Cell viability, viable cell density and expression of the protein of interest are improved relative to cells grown in culture without spermine or spermidine at a concentration of at least about 0.10 μM, or putrescine at a concentration of at least about 100 μM. In some embodiments are provided mammalian cell lines, other embodiments provide Chinese Hamster Ovary (CHO) cell lines. Also provided are serum-free media that are also peptone-free.

The present invention also provides a cell culture comprising an animal cell line expressing a protein of interest in serum free cell culture medium comprising spermine and/or spermidine at a concentration of at least about 0.10 μM, and/or putrescine at a concentration of at least about 100 μM.

Within the present invention is the serum-free cell culture media comprising putrescine at a concentration of at least about 100 to at least about 1000 μM. Also included are serum free cell culture media comprising spermidine at a concentration of at least about 0.10 μM to at least about 500 μM. In some embodiments spermidine concentration is at least about 10 μM to at least about 200 μM. In some embodiments spermidine concentration is at least about 10 μM to at least about 50 μM. The invention further provides serum free cell culture media comprising spermine at a concentration of at least about 0.10 μM to at least about 500 μM. In some embodiments spermine concentration is at least about 10 μM to at least about 200 μM. In some embodiments spermine concentration is at least about 10 μM to at least about 50 μM. In some embodiments spermine concentration is at least about 50 μM to at least about 200 μM. In some embodiments spermine concentration is at least about 50 μM.

Within embodiments of the invention the protein of interest is an antibody, a human antibody, a humanized antibody, a chimeric antibody, a monoclonal antibody, a multispecific antibody, an antigen binding antibody fragment, a single chain antibody, a diabody, triabody or tetrabody, a Fab fragment or a F(ab′)₂ fragment, an IgD antibody, an IgE antibody, an IgM antibody, an IgG antibody, an IgG1 antibody, an IgG2 antibody, an IgG3 antibody, and an IgG4 antibody. In one embodiment, the antibody is an IgG1 antibody. In another embodiment, the antibody is an IgG2 antibody.

The invention provides for the use of a concentrated feed medium comprising spermine or spermidine at a concentration such that when added to the cell culture the concentration of spermidine and/or spermine is at least about 0.10 μM. Some concentrated feed medium comprise putrescine at a concentration such that when added to the cell culture the concentration of putrescine is of at least about 100 μM.

### BRIEF DESCRIPTION OF THE FIGURES

FIG. 1a shows viable cell density measured after three days of culture in DMEM/F12 with or without supplemental polyamines. Viable cell density was higher in cultures with supplemental spermine (smn), spermidine (smd), and putrescine (put) than cells grown in the basal DMEM/F12 media. The greatest increase in viable cell density was seen with supplemental spermine.

FIG. 1b shows culture viability measured after three days of culture in DMEM/F12 with or without supplemental polyamines. Viability was higher in cultures with supplemented with spermine (smn), spermidine (smd), and putrescine (put) than cells without supplemental polyamines. The greatest increase in cell viability was seen at a spermine concentration of 50 μM.

FIG. 2a shows viable cell density measured after six days in culture in VM-Soy with and without supplemental spermine. The greatest increase in viable cell density was seen at a spermine concentration of 50 μM. The error bars show the range of data.

FIG. 2b shows culture viability measured after six days in culture in VM-Soy with and without supplemental spermine. The greatest increase in cell viability was seen at spermine concentrations of between 50 μM and 200 μM. The error bars show the range of data.

FIG. 2c shows recombinant antibody concentration after six days in culture in VM-Soy with and without supplemental spermine. The greatest increase in antibody concentration was seen at a spermine concentration of 50 μM. The error bars show the range of data.

FIG. 3a shows the viable cell density at different days during a fed-batch production culture. Each graph shows the viable cell density of a different cell line. Solid symbols represent cultures supplemented with 10 μM spermine and open symbols represent controls without spermine. Each graph is shown as an average of duplicate cultures. Error bars show the range of the data. Graph 1 shows the viable cell density for a cell line expressing IgG₂ antibody A. Graph 2 shows the viable cell density for a cell line expressing IgG₂ antibody B. Graph 3 shows the viable cell density for a cell line expressing IgG antibody C. Graph 4 shows the viable cell density for a cell line expressing IgG₂ antibody D.

FIG. 3b shows the cell viability at different days during the fed-batch production culture. Each graph shows the viability of a different cell line. Solid symbols represent cultures supplemented with 10 μM spermine and open symbols represent controls without spermine. Each graph is shown as an average of duplicate cultures. Error bars show the range of the data. Graph 1 shows the viability for a cell line expressing IgG₂ antibody A. Graph 2 shows the viability for a cell line expressing IgG₂ antibody B. Graph 3 shows the viability for a cell line expressing IgG₂ antibody C. Graph 4 shows the viability for a cell line expressing IgG₂ antibody D.

FIG. 3c shows the relative antibody titers on different days during the fed-batch production culture. The reported values are relative to the titer produced by the control cells on Day 11. Each graph shows the relative antibody titer of a different cell line. Solid symbols represent cultures supplemented with 10 μM spermine and open symbols represent controls without spermine. Each graph is shown as an average of duplicate cultures. Error bars show the range of the data. Graph 1 shows the relative titers for a cell line expressing IgG₂ antibody A. Graph 2 shows the relative titers for a cell line expressing IgG2 antibody B. Graph 3 shows the relative titers for a cell line expressing IgG₂ antibody C. Graph 4 shows the relative titers for a cell line expressing IgG₂ antibody D.

FIG. 4a shows the viable cell density of each spermine-treated culture at different days during the fed-batch production process. The data are shown as an average of duplicate cultures. Error bars show the range of the data.

FIG. 4b compares the viable cell density of spermine-treated cultures on Day 8 of culture (ending viable cell density) normalized to the control culture.

FIG. 4c shows the ending cell viability of each culture on Day 8 the fed-batch production process. The data are shown as an average of duplicate cultures. Error bars show the range of the data.

FIG. 4d shows the relative antibody titers on Day 8 of fed-batch production. The reported values are relative to the titer produced by the control cells on Day 8. The data is shown as an average of duplicate cultures. Error bars show the range of the data.

### DETAILED DESCRIPTION OF THE INVENTION

While the terminology used in this application is standard within the art, definitions of certain terms are provided herein to assure clarity and definiteness to the meaning of the claims. Units, prefixes, and symbols may be denoted in their SI accepted form. Numeric ranges recited herein are inclusive of the numbers defining the range and include and are supportive of each integer within the defined range. Unless otherwise noted, the terms “a” or “an” are to be construed as meaning “at least one of”. The section headings used herein are for organizational purposes only and are not to be construed as limiting the subject matter described. The methods and techniques described herein are generally performed according to conventional methods well known in the art and as described in various general and more specific references that are cited and discussed throughout the present specification unless otherwise indicated. See, e.g., Sambrook et al. Molecular Cloning: A Laboratory Manual, 3rd ed., Cold Spring Harbor Laboratory Press, Cold Spring Harbor, N.Y. (2001) and Ausubel et al., Current Protocols in Molecular Biology, Greene Publishing Associates (1992), and Harlow and Lane Antibodies: A Laboratory Manual Cold Spring Harbor Laboratory Press, Cold Spring Harbor, N.Y. (1990). All documents, or portions of documents, cited in this application, including but not limited to patents, patent applications, articles, books, and treatises, are hereby expressly incorporated by reference.

As used herein “peptide,” “polypeptide” and “protein” are used interchangeably throughout and refer to a molecule comprising two or more amino acid residues joined to each other by peptide bonds. Peptides, polypeptides and proteins are also inclusive of modifications including, but not limited to, glycosylation, lipid attachment, sulfation, gamma-carboxylation of glutamic acid residues, hydroxylation and ADP-ribosylation. Polypeptides can be of scientific or commercial interest, including protein-based drugs. Polypeptides include, among other things, antibodies and chimeric or fusion proteins. Polypeptides are produced by recombinant animal cell lines using cell culture methods.

The term “antibody” includes reference to both glycosylated and non-glycosylated immunoglobulins of any isotype or subclass or to an antigen-binding region thereof that competes with the intact antibody for specific binding, unless otherwise specified, including human, humanized, chimeric, multi-specific, monoclonal, polyclonal, and oligomers or antigen binding fragments thereof. Antibodies can be any class of immunoglobulin. Also included are proteins having an antigen binding fragment or region such as Fab, Fab′, F(ab′)₂, Fv, diabodies, Fd, dAb, maxibodies, single chain antibody molecules, complementarity determining region (CDR) fragments, scFv, diabodies, triabodies, tetrabodies and polypeptides that contain at least a portion of an immunoglobulin that is sufficient to confer specific antigen binding to a target polypeptide. The term “antibody” is inclusive of, but not limited to, those that are prepared, expressed, created or isolated by recombinant means, such as antibodies isolated from a host cell transfected to express the antibody.

Chimeric or fusion proteins are inclusive of, but not limited to, Fc fusion proteins comprising part or all of two or more proteins, one of which is an Fc portion of an immunoglobulin molecule, that are not fused in their natural state. Preparation of fusion proteins comprising certain heterologous polypeptides fused to various portions of antibody-derived polypeptides (including the Fc domain) has been described, e.g., by Ashkenazi et al., Proc. Natl. Acad. Sci. USA 88:10535, 1991; Byrn et al., Nature 344:677, 1990; and Hollenbaugh et al., “Construction of Immunoglobulin Fusion Proteins”, in Current Protocols in Immunology, Suppl. 4, pages 10.19.1-10.19.11, 1992. Examples of such Fc fusion proteins include, but are not limited to, human receptor activator of NF-KappaB fused to an Fc portion of an immunoglobulin molecule (huRANK:Fc), tunica internal endothelial cell kinase-delta fused to an Fc portion of an immunoglobulin molecule (TEKdelta:Fc) and tumor necrosis factor receptor fused to an Fc portion of an immunoglobulin molecule (TNFR:Fc).

The invention is based, in part, on the discovery that addition of polyamines to serum free cell culture media results in increased cell growth, viability and polypeptide production from a recombinantly engineered animal cell line expressing a protein of interest, thereby enhancing culture robustness, improving the yield of the polypeptide of interest.

The present invention provides a method comprising culturing an animal cell line expressing a protein of interest in serum free cell culture medium; the medium comprising spermine or spermidine at a concentration of at least about 0.1 μM, or putrescine at a concentration of at least about 100 μM. The medium may also comprise combinations of spermine, spermidine and putrescine together at these concentrations. Cell viability, viable cell density and/or expression of the protein of interest are improved relative to cells grown in culture without spermine or spermidine at a concentration of at least about 0.1 μM, or putrescine at a concentration of at least about 100 μM. Some animal cell lines are mammalian cell lines. Mammalian cell lines are inclusive of, but not limited to, Chinese Hamster Ovary (CHO) cell lines. Some serum-free media are also peptone-free media.

The present invention also provides a cell culture comprising an animal cell line expressing a protein of interest in serum free cell culture medium comprising spermine and/or spermidine at a concentration of at least about 0.1 μM, and/or putrescine at a concentration of at least about 100 μM.

Within the present invention is the addition to serum free cell culture media of putrescine at concentrations of at least about 100 μM. Some serum free cell culture media comprise putrescine at a concentration of at least about 100 to at least about 1000 μM. Other serum free media include those comprising a putrescine concentration of at least about 100, 150, 200, 250, 300, 350, 400, 450, 500, 500, 600, 650, 700, 750, 800, 850, 900, 950, or 1000 μM.

Also within the present invention is the addition to serum free cell culture media of spermidine at concentrations of at least about 0.10 μM. Some serum free cell culture media comprise spermidine at a concentration of at least about 0.10 μM to at least about 500 μM. Some serum free cell culture media comprise spermidine at a concentration of at least about 10 μM to at least about 500 μM. Other serum free media includes those comprising spermidine at a concentration of at least about 10 μM to at least about 200 μM. Other serum free media includes those comprising spermidine at a concentration of at least about 10 μM to at least about 100 μM. Other serum free media includes those comprising spermidine at a concentration of at least about 10 μM to at least about 50 μM. Other serum free media include those comprising a spermidine concentration of at least about 10, 15, 20, 25, 30, 35, 40, 45, 50, 100, 200, 300, 400, or 500 μM.

The present invention also provides the addition to serum free cell culture media of spermine at concentrations of at least about 0.10 μM. Some serum free cell culture media comprise spermine at a concentration of at least about 10 μM. Some serum free cell culture media comprise spermine at a concentration of at least about 0.10 μM to at least about 500 μM. Other serum free media includes those comprising spermine at a concentration of at least about 10 μM to at least about 500 μM. Other serum free media includes those comprising spermine at a concentration of at least about 10 μM to at least about 200 μM. Other serum free media includes those comprising spermine at a concentration of at least about 10 μM to at least about 100 μM. Some serum free media includes those comprising spermine at a concentration of at least about 10 μM to at least about 50 μM. Additional serum free cell culture media comprise spermine at a concentration of at least about 50 μM to at least about 200 μM. Additional serum free cell culture media comprise spermine at a concentration of at least about 50 μM to at least about 500 μM. Still other serum free cell culture comprise spermine at a concentration of at least about 0.1, 0.5, 1.0, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 250, 300, 350, 400, 450, or 500 μM.

Within embodiments of the invention the protein of interest is an antibody, a human antibody, a humanized antibody, a chimeric antibody, a monoclonal antibody, a multispecific antibody, an antigen binding antibody fragment, a single chain antibody, a diabody, triabody or tetrabody, a Fab fragment or a F(ab′)₂ fragment, an IgD antibody, an IgE antibody, an IgM antibody, an IgG antibody, an IgG1 antibody, an IgG2 antibody, an IgG3 antibody, or an IgG4 antibody. In one embodiment, the antibody is an IgG1 antibody. In one embodiment, the antibody is an IgG2 antibody.

The invention provides use of a concentrated feed medium comprising spermidine and/or spermine such that the concentration of spermidine and/or spermine when added to the cell culture is of at least about 0.1 μM. Some concentrated feed medium comprise putrescine at a concentration such that the concentration of putrescine when added to the cell culture is of at least about 100 μM.

The polyamines of the present invention may be added to a basal or enriched cell culture media. The polyamines may be added to a prepared cell culture media, such as a commercial media, or the polyamines can be added at the time the cell culture media is prepared. The polyamines may also be added to an existing cell culture via separate stock solutions, added at any time during the cell culture. The polyamines may also be added to concentrated feed media such that the concentration of spermine or spermidine in the cell culture is of at least about 0.10 μM, or such that the concentration of putrescine when added to the cell culture is of at least about 100 μM.

Polyamines such as putrescine, spermidine and spermine are commercially available from a number of vendors such as Sigma-Adrich (St. Louis, Mo.); EMD/Calbiochem (San Diego, Calif.); Alexis Biochemicals (San Diego, Calif.).

For the purposes of this invention, cell culture medium is a media suitable for growth of animal cells, such as mammalian cells, in in vitro cell culture. Cell culture media formulations are well known in the art. Typically, cell culture media are comprised of buffers, salts, carbohydrates, amino acids, vitamins and trace essential elements. The cell culture medium may or may not contain serum, peptone, and/or proteins. Various tissue culture media, including serum-free and defined culture media, are commercially available, for example, any one or a combination of the following cell culture media can be used: RPMI-1640 Medium, RPMI-1641 Medium, Dulbecco's Modified Eagle's Medium (DMEM), Minimum Essential Medium Eagle, F-12K Medium, Ham's F12 Medium, Iscove's Modified Dulbecco's Medium, McCoy's 5A Medium, Leibovitz's L-15 Medium, and serum-free media such as EX-CELL™ 300 Series (JRH Biosciences, Lenexa, Kans.), among others. Cell culture media may be supplemented with additional or increased concentrations of components such as amino acids, salts, sugars, vitamins, hormones, growth factors, buffers, antibiotics, lipids, trace elements and the like, depending on the requirements of the cells to be cultured and/or the desired cell culture parameters. For example, cell culture media may be supplemented with polyamines such as putrescine, spermidine and spermine, to improve cell growth, cell viability, and/or recombinant protein production in association with a particular host cell.

Cell culture media may be serum-free, protein-free, and/or peptone-free media. “Serum-free” applies to a cell culture medium that does not contain animal sera, such as fetal bovine serum. “Protein-free” applies to cell culture media free from exogenously added protein, such as transferring, protein growth factors IGF-1, or insulin. Protein-free media may or may not contain peptones. “Peptone-free” applies to cell culture media which contains no exogenous protein hydrolysates such as animal and/or plant protein hydrolysates. Eliminating serum and/or hydrolysates from cell culture media has the advantage of reducing lot to lot variability and enhancing processing steps, such as filtration. However, when serum and/or peptone are removed from the cell culture media, cell growth, viability and/or protein expression may be diminished or less than optimal. As such, serum-free and/or peptone-free cell culture medium may be highly enriched for amino acids, trace elements and the like. See, for example, U.S. Pat. Nos. 5,122,469 and 5,633,162. Although there are many media formulations, there is a need to develop defined media formulations that perform as well or preferably better than those containing animal sera and/or peptones.

Defined cell culture formulations are complex, containing amino acids, inorganic salts, carbohydrates, lipids, vitamins, buffers and trace essential elements. Identifying the components that are necessary and beneficial to maintain a cell culture with desired characteristics is an on going task. Defined basal media formulations which are supplemented or enriched to meet the needs of a particular host cell or to meet desired performance parameters is one approach to developing defined media. Identifying those components and optimum concentrations that lead to improved cell growth, viability and protein production is an ongoing task.

The polyamines, putrescine, spermidine and spermine, have been implicated in a variety of physiological and pathophysiological processes, their role in cell growth, differentiation and cell death is still not completely understood (Jänne et al., (2004) Eur. J. Biochem. 271: 87-894; Wallace et al., (2003) Biochem J. 376: 1-14). Of the polyamines, putrescine has been included, at very low concentrations, as a component in some cell culture media formulations; see for example WO 2005/028626 (0.02-0.08 mg/l putrescine); U.S. Pat. No. 5,426,699 (0.08 mg/l); U.S. Pat. No. RE30,985 (0.16 mg/l); U.S. Pat. No. 5,811,299 (0.27 mg/l); U.S. Pat. No. 5,122,469 (0.5635 mg/l); U.S. Pat. No. 5,063,157 (1 mg/l).

High exogenous polyamine concentrations, particularly high spermine concentrations (2 mM), have been shown to contribute to the inhibition of cell growth and/or result in cell death (Brunton et al., (1990) Biochem. Pharmacol. 40: 1893-1990; Brunton et al., (1991) Biochem J. 280: 193-198). Human-human hybridoma HB4C5 cells grown in media supplemented with spermine concentrations of 2.4 mM or higher suppressed cell growth, viability declined after only 3 days and protein production, while increased over the control, was at less than 1 mg/l (Miyazaki et al., (1998) Cytotechnology 26:111-118). Completely protein free clonal growth of Chinese Hamster CHD-3 cells was obtained in media containing linoleic acid and putrescine, spermidine or spermine as measured by colony size (Ham, (1964) Biocheim. Biophys. Res. Comm. 14: 34-38). These media were not optimized to increase recombinant protein production while maintaining cell growth and viability. The cells were not genetically engineered to express proteins of interest nor were the media optimized for high density, large scale growth of cell lines genetically engineered to produce and/or secrete proteins of commercial interest. Such media may be expensive to use and/or laborious to prepare or require additional supplements to achieve suitable sustained growth of genetically engineered cell lines.

By cell culture or “culture” is meant the growth and propagation of cells outside of a multicellular organism or tissue. Suitable culture conditions for mammalian cells are known in the art. See e.g. Animal cell culture: A Practical Approach, D. Rickwood, ed., Oxford University Press, New York (1992). Mammalian cells may be cultured in suspension or while attached to a solid substrate. Fluidized bed bioreactors, hollow fiber bioreactors, roller bottles, shake flasks, or stirred tank bioreactors, with or without microcarriers, and operated in a batch, fed batch, continuous, semi-continuous, or perfusion mode are available for mammalian cell culture. Cell culture media and/or concentrated feed media may be added to the culture continuously or at intervals during the culture. For example, a culture may be fed once per day, every other day, every three days, or may be fed when the concentration of a specific medium component, which is being monitored, falls outside a desired range.

Animal cells, such as CHO cells, may be cultured in small scale cultures, such as for example, in 100 ml containers having about 30 ml of media, 250 ml containers having about 80 to about 90 ml of media, 250 ml containers having about 150 to about 200 ml of media. Alternatively, the cultures can be large scale such as for example 1000 ml containers having about 300 to about 1000 ml of media, 3000 ml containers having about 500 ml to about 3000 ml of media, 8000 ml containers having about 2000 ml to about 8000 ml of media, and 15000 ml containers having about 4000 ml to about 15000 ml of media.

Large scale cell cultures, such as for clinical manufacturing of protein therapeutics, are typically maintained for days, or even weeks, while the cells produce the desired protein(s). During this time the culture can be supplemented with a concentrated feed medium containing components, such as nutrients and amino acids, which are consumed during the course of the culture. Concentrated feed medium may be based on just about any cell culture media formulation. Such a concentrated feed medium can contain most of the components of the cell culture medium at, for example, about 5×, 6×, 7×, 8×, 9×, 10×, 12×, 14×, 16×, 20×, 30×, 50×, 100×, 200×, 400×, 600×, 800×, or even about 1000× of their normal amount. Concentrated feed media are often used in fed batch culture processes.

The methods according to the present invention may be used to improve the production of recombinant proteins in both single phase and multiple phase culture processes. In a single phase process, cells are inoculated into a culture environment and the disclosed methods are employed during the single production phase. In a multiple stage process, cells are cultured in two or more distinct phases. For example cells may be cultured first in one or more growth phases, under environmental conditions that maximize cell proliferation and viability, then transferred to a production phase, under conditions that maximize protein production. In a commercial process for production of a protein by mammalian cells, there are commonly multiple, for example, at least about 2, 3, 4, 5, 6, 7, 8, 9, or 10 growth phases that occur in different culture vessels preceding a final production phase. The growth and production phases may be preceded by, or separated by, one or more transition phases. In multiple phase processes, the methods according to the present invention can be employed at least during the production phase, although they may also be employed in a preceding growth phase. A production phase can be conducted at large scale. A large scale process can be conducted in a volume of at least about 100, 500, 1000, 2000, 3000, 5000, 7000, 8000, 10,000, 15,000, 20,000 liters. A growth phase may occur at a higher temperature than a production phase. For example, a growth phase may occur at a first temperature from about 35° C. to about 38° C., and a production phase may occur at a second temperature from about 29° C. to about 37° C., optionally from about 30° C. to about 36° C. or from about 30° C. to about 34° C. In addition, chemical inducers of protein production, such as, for example, caffeine, butyrate, and hexamethylene bisacetamide (HMBA), may be added at the same time as, before, and/or after a temperature shift. If inducers are added after a temperature shift, they can be added from one hour to five days after the temperature shift, optionally from one to two days after the temperature shift.

The invention finds particular utility in improving cell growth, viability and/or protein production via cell culture processes. The cell lines (also referred to as “host cells”) used in the invention are genetically engineered to express a polypeptide of commercial or scientific interest. Cell lines are typically derived from a lineage arising from a primary culture that can be maintained in culture for an unlimited time. Genetically engineering the cell line involves transfecting, transforming or transducing the cells with a recombinant polynucleotide molecule, and/or otherwise altering (e.g., by homologous recombination and gene activation or fusion of a recombinant cell with a non-recombinant cell) so as to cause the host cell to express a desired recombinant polypeptide. Methods and vectors for genetically engineering cells and/or cell lines to express a polypeptide of interest are well known to those of skill in the art; for example, various techniques are illustrated in Current Protocols in Molecular Biology, Ausubel et al., eds. (Wiley & Sons, New York, 1988, and quarterly updates); Sambrook et al., Molecular Cloning: A Laboratory Manual (Cold Spring Laboratory Press, 1989); Kaufman, R. J., Large Scale Mammalian Cell Culture, 1990, pp. 15-69.

Animal cell lines are derived from cells whose progenitors were derived from a multi-cellular animal. One type of animal cell line is a mammalian cell line. A wide variety of mammalian cell lines suitable for growth in culture are available from the American Type Culture Collection (Manassas, Va.) and commercial vendors. Examples of cell lines commonly used in the industry include VERO, BHK, HeLa, CV1 (including Cos), MDCK, 293, 3T3, myeloma cell lines (e.g., NSO, NS1), PC12, WI38 cells, and Chinese hamster ovary (CHO) cells. CHO cells are widely used for the production of complex recombinant proteins, e.g. cytokines, clotting factors, and antibodies (Brasel et al. (1996), Blood 88:2004-2012; Kaufman et al. (1988), J. Biol Chem 263:6352-6362; McKinnon et al. (1991), J Mol Endocrinol 6:231-239; Wood et al. (1990), J. Immunol. 145:3011-3016). The dihydrofolate reductase (DHFR)-deficient mutant cell lines (Urlaub et al. (1980), Proc Natl Acad Sci USA 77: 4216-4220), DXB11 and DG-44, are desirable CHO host cell lines because the efficient DHFR selectable and amplifiable gene expression system allows high level recombinant protein expression in these cells (Kaufman R. J. (1990), Meth Enzymol 185:537-566). In addition, these cells are easy to manipulate as adherent or suspension cultures and exhibit relatively good genetic stability. CHO cells and proteins recombinantly expressed in them have been extensively characterized and have been approved for use in clinical commercial manufacturing by regulatory agencies.

The methods of the invention can be used to culture cells that express a protein(s) of interest. The expressed protein(s) may be produced intracellularly or be secreted into the culture medium from which they can be recovered and/or collected. In addition, the protein(s) can be purified, or partially purified, from such culture or component (e.g., from culture medium or cell extracts or bodily fluid) using known processes and products available from commercial vendors. The purified protein(s) can then be “formulated”, meaning buffer exchanged, sterilized, bulk-packaged, and/or packaged for a final user. Suitable formulations for pharmaceutical compositions include those described in Remington's Pharmaceutical Sciences, 18th ed. 1995, Mack Publishing Company, Easton, Pa.

Examples of polypeptides that can be produced with the methods of the invention include proteins comprising amino acid sequences identical to or substantially similar to all or part of one of the following proteins: a flt3 ligand (WO 94/28391), a CD40 ligand (U.S. Pat. No. 6,087,329), erythropoietin, thrombopoietin, calcitonin, leptin, IL-2, angiopoietin-2 (Maisonpierre et al. (1997), Science 277(5322): 55-60), Fas ligand, ligand for receptor activator of NF-kappa B (RANKL, WO 01/36637), tumor necrosis factor (TNF)-related apoptosis-inducing ligand (TRAIL, WO 97/01633), thymic stroma-derived lymphopoietin, granulocyte colony stimulating factor, granulocyte-macrophage colony stimulating factor (GM-CSF, Australian Patent No. 588819), mast cell growth factor, stem cell growth factor (U.S. Pat. No. 6,204,363), epidermal growth factor, keratinocyte growth factor, megakaryote growth and development factor, RANTES, human fibrinogen-like 2 protein (FGL2; NCBI accession no. NM_{—}00682; Ruegg and Pytela (1995), Gene 160:257-62) growth hormone, insulin, insulinotropin, insulin-like growth factors, parathyroid hormone, interferons including α-interferons, γ-interferon, and consensus interferons (U.S. Pat. Nos. 4,695,623 and 4,897471), nerve growth factor, brain-derived neurotrophic factor, synaptotagmin-like proteins (SLP 1-5), neurotrophin-3, glucagon, interleukins, colony stimulating factors, lymphotoxin-β, tumor necrosis factor (TNF), leukemia inhibitory factor, oncostatin-M, and various ligands for cell surface molecules ELK and Hek (such as the ligands for eph-related kinases or LERKS). Descriptions of proteins that can be produced according to the inventive methods may be found in, for example, Human Cytokines: Handbook for Basic and Clinical Research, all volumes (Aggarwal and Gutterman, eds. Blackwell Sciences, Cambridge, Mass., 1998); Growth Factors: A Practical Approach (McKay and Leigh, eds., Oxford University Press Inc., New York, 1993); and The Cytokine Handbook, Vols. 1 and 2 (Thompson and Lotze eds., Academic Press, San Diego, Calif., 2003).

Additionally the methods of the invention would be useful to produce proteins comprising all or part of the amino acid sequence of a receptor for any of the above-mentioned proteins, an antagonist to such a receptor or any of the above-mentioned proteins, and/or proteins substantially similar to such receptors or antagonists. These receptors and antagonists include: both forms of tumor necrosis factor receptor (TNFR, referred to as p55 and p75, U.S. Pat. No. 5,395,760 and U.S. Pat. No. 5,610,279), Interleukin-1 (IL-1) receptors (types I and II; EP Patent No. 0460846, U.S. Pat. No. 4,968,607, and U.S. Pat. No. 5,767,064,), IL-1 receptor antagonists (U.S. Pat. No. 6,337,072), antagonists or inhibitors (U.S. Pat. Nos. 5,981,713, 6,096,728, and 5,075,222) IL-2 receptors, IL-4 receptors (EP Patent No. 0 367 566 and U.S. Pat. No. 5,856,296), IL-15 receptors, IL-17 receptors, IL-18 receptors, Fc receptors, granulocyte-macrophage colony stimulating factor receptor, granulocyte colony stimulating factor receptor, receptors for oncostatin-M and leukemia inhibitory factor, receptor activator of NF-kappa B (RANK, WO 01/36637 and U.S. Pat. No. 6,271,349), osteoprotegerin (U.S. Pat. No. 6,015,938), receptors for TRAIL (including TRAIL receptors 1, 2, 3, and 4), and receptors that comprise death domains, such as Fas or Apoptosis-Inducing Receptor (AIR).

Other proteins that can be produced using the invention include proteins comprising all or part of the amino acid sequences of differentiation antigens (referred to as CD proteins) or their ligands or proteins substantially similar to either of these. Such antigens are disclosed in Leukocyte Typing VI (Proceedings of the VIth International Workshop and Conference, Kishimoto, Kikutani et al., eds., Kobe, Japan, 1996). Similar CD proteins are disclosed in subsequent workshops. Examples of such antigens include CD22, CD27, CD30, CD39, CD40, and ligands thereto (CD27 ligand, CD30 ligand, etc.). Several of the CD antigens are members of the TNF receptor family, which also includes 41BB and OX40. The ligands are often members of the TNF family, as are 41BB ligand and OX40 ligand.

Enzymatically active proteins or their ligands can also be produced using the invention. Examples include proteins comprising all or part of one of the following proteins or their ligands or a protein substantially similar to one of these: a disintegrin and metalloproteinase domain family members including TNF-alpha Converting Enzyme, various kinases, glucocerebrosidase, superoxide dismutase, tissue plasminogen activator, Factor VIII, Factor IX, apolipoprotein E, apolipoprotein A-I, globins, an IL-2 antagonist, alpha-1 antitrypsin, ligands for any of the above-mentioned enzymes, and numerous other enzymes and their ligands.

The invention can also be used to produce antibodies or portions thereof. Such antibodies can include conjugates comprising an antibody and a cytotoxic or luminescent substance. Such substances include: maytansine derivatives (such as DM1); enterotoxins (such as a Staphlyococcal enterotoxin); iodine isotopes (such as iodine-125); technium isotopes (such as Tc-99m); cyanine fluorochromes (such as Cy5.5.18); and ribosome-inactivating proteins (such as bouganin, gelonin, or saporin-S6). Examples of antibodies include, but are not limited to, those that recognize any one or a combination of proteins including, but not limited to, the above-mentioned proteins and/or the following antigens: CD2, CD3, CD4, CD8, CD11a, CD14, CD18, CD20, CD22, CD23, CD25, CD33, CD40, CD44, CD52, CD80 (B7.1), CD86 (B7.2), CD147, IL-α, IL-β, IL-2, IL-3, IL-7, IL-4, IL-5, IL-8, IL-10, IL-2 receptor, IL-4 receptor, IL-6 receptor, IL-13 receptor, IL-18 receptor subunits, FGL2, PDGF-β and analogs thereof (see U.S. Pat. Nos. 5,272,064 and 5,149,792), VEGF, TGF, TGF-β2, TGF-β1, EGF receptor (see U.S. Pat. No. 6,235,883) VEGF receptor, hepatocyte growth factor, osteoprotegerin ligand, interferon gamma, B lymphocyte stimulator (BlyS, also known as BAFF, THANK, TALL-1, and zTNF4; see Do and Chen-Kiang (2002), Cytokine Growth Factor Rev. 13(1): 19-25), C5 complement, IgE, tumor antigen CA125, tumor antigen MUC1, PEM antigen, LCG (which is a gene product that is expressed in association with lung cancer), HER-2, a tumor-associated glycoprotein TAG-72, the SK-1 antigen, tumor-associated epitopes that are present in elevated levels in the sera of patients with colon and/or pancreatic cancer, cancer-associated epitopes or proteins expressed on breast, colon, squamous cell, prostate, pancreatic, lung, and/or kidney cancer cells and/or on melanoma, glioma, or neuroblastoma cells, the necrotic core of a tumor, integrin alpha 4 beta 7, the integrin VLA-4, B2 integrins, TRAIL receptors 1, 2, 3, and 4, RANK, RANK ligand, TNF-α, the adhesion molecule VAP-1, epithelial cell adhesion molecule (EpCAM), intercellular adhesion molecule-3 (ICAM-3), leukointegrin adhesin, the platelet glycoprotein gp IIb/IIIa, cardiac myosin heavy chain, parathyroid hormone, rNAPc2 (which is an inhibitor of factor VIIa-tissue factor), MHC 1, carcinoembryonic antigen (CEA), alpha-fetoprotein (AFP), tumor necrosis factor (TNF), CTLA-4 (which is a cytotoxic T lymphocyte-associated antigen), Fc-γ-1 receptor, HLA-DR 10 beta, HLA-DR antigen, L-selectin, Respiratory Syncitial Virus, human immunodeficiency virus (HIV), hepatitis B virus (HBV), Streptococcus mutans, and Staphlycoccus aureus.

The invention may also be used to produce all or part of an anti-idiotypic antibody or a substantially similar protein, including anti-idiotypic antibodies against: an antibody targeted to the tumor antigen gp72; an antibody against the ganglioside GD3; an antibody against the ganglioside GD2; or antibodies substantially similar to these.

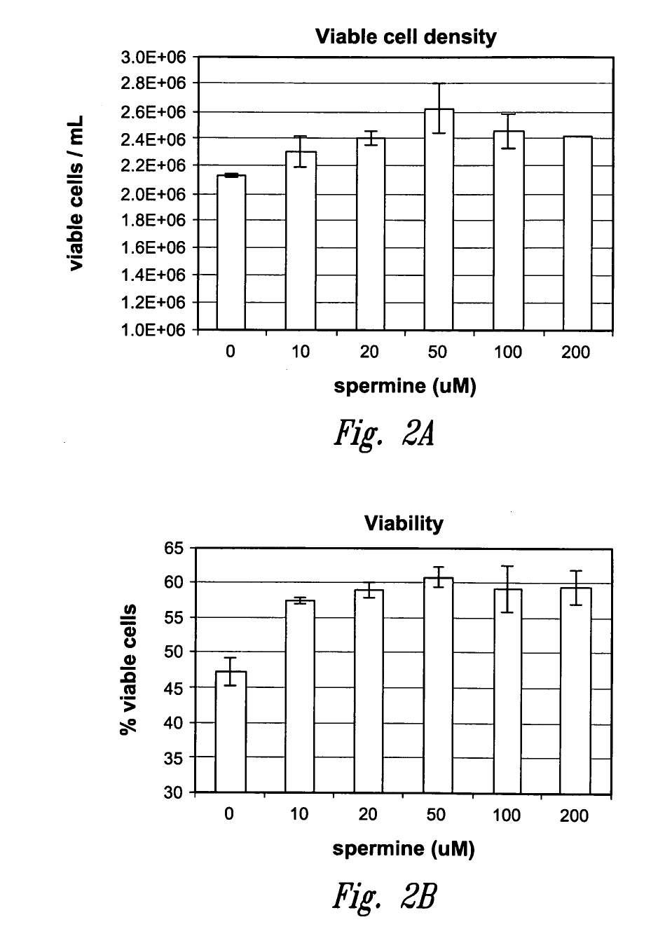

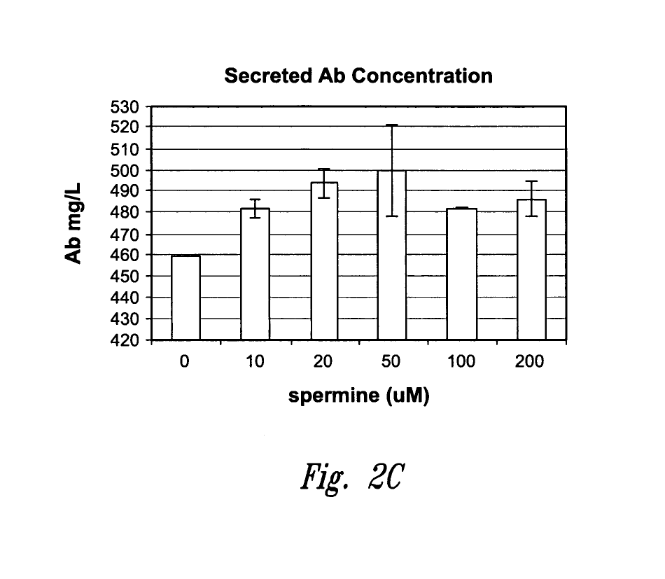

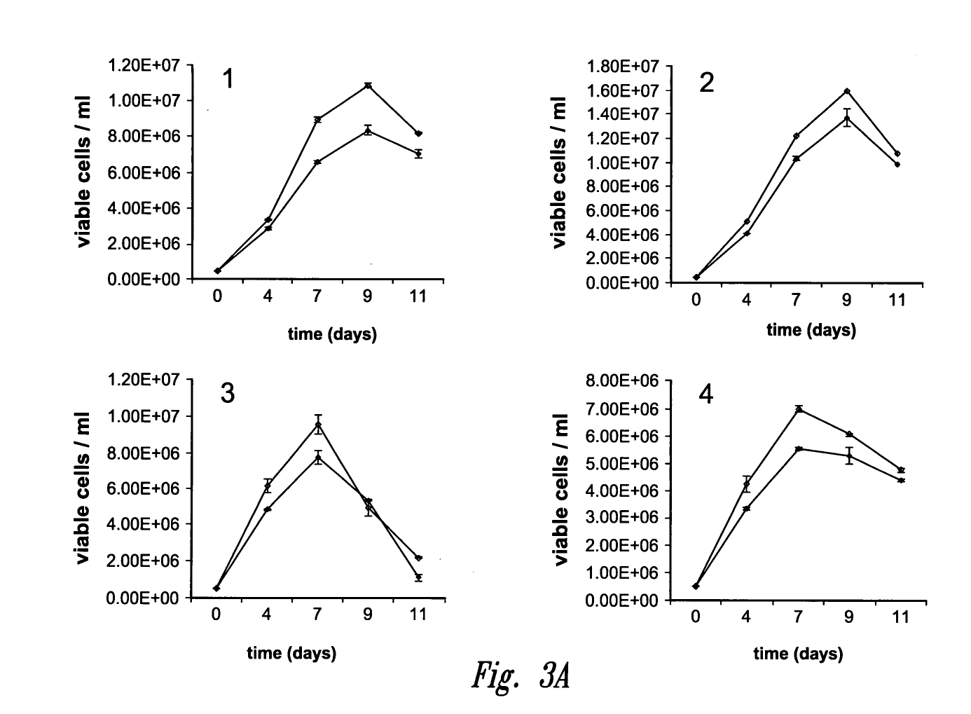

The invention can also be used to produce recombinant fusion proteins comprising, for example, any of the above-mentioned proteins. For example, recombinant fusion proteins comprising one of the above-mentioned proteins plus a multimerization domain, such as a leucine zipper, a coiled coil, an Fc portion of an immunoglobulin, or a substantially similar protein, can be produced using the methods of the invention. See e.g. WO94/10308; Lovejoy et al. (1993), Science 259:1288-1293; Harbury et al. (1993), Science 262:1401-05; Harbury et al. (1994), Nature 371:80-83; Håkansson et al. (1999), Structure 7:255-64. Specifically included among such recombinant fusion proteins are proteins in which a portion of TNFR or RANK is fused to an Fc portion of an antibody (TNFR:Fc or RANK:Fc). TNFR:Fc comprises the Fc portion of an antibody fused to an extracellular domain of TNFR, which includes amino acid sequences substantially similar to amino acids 1-163, 1-185, or 1-235 of FIG. 2A of U.S. Pat. No. 5,395,760. RANK:Fc is described in International Application WO 01/36637.

The present invention is not to be limited in scope by the specific embodiments described herein that are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

### EXAMPLES

### Example 1

To test the effect of high concentration polyamine media formulations on cell culture performance a CHO cell line producing a recombinant IgG₂ monoclonal antibody was seeded at 2.5×10⁵ cells/ml in serum-free DMEM/F12 (SAFC, Lenexa, Kans.). DMEM/F12 contains 503 nM putrescine as a component of the commercial formulation. The media was supplemented with an additional 100-1000 μM putrescine dihydrochloride (Sigma-Aldrich, St. Louis, Mo.); 10-50 μM spermidine tetrachdrochloride (Sigma-Aldrich), or 10-50 μM spermine tetrahydrochloride (Sigma-Aldrich). Cells were maintained in suspension culture for three days at 36° C. in 5% CO₂. Total cell density and viable cell density were measured using a Guava Easy-Cyte™ flow cytometer and Guava Viacount® Flex reagent (Hayward, Calif.) according to manufacture's instructions.

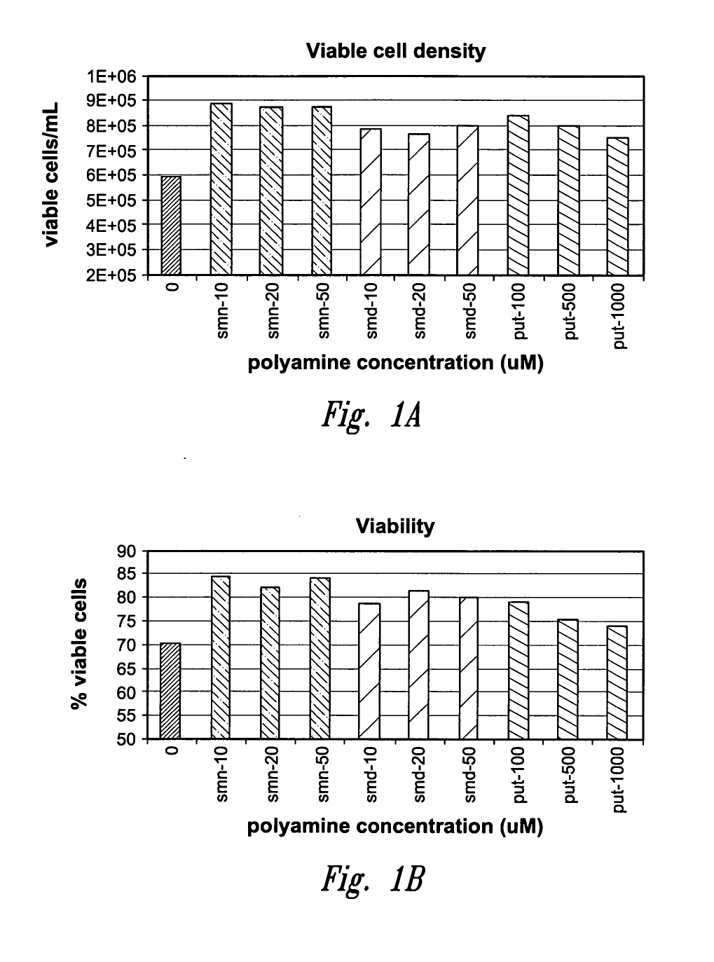

Following three days culture in the polyamine-supplemented-DMEM/F12 media, viable cell density and culture viability were measured, see FIGS. 1a and 1b. Mean viability is presented as percent of viable cells and viable cell density is presented as number of cells/ml. Both viability and viable cell density were greater in those cultures supplemented with spermine (smn), spermidine (smd), and additional putrescine (put) compared to the unsupplemented control media. Viable cell density increased between 27-49% and cell viability increased between 5-20%, compared to the unsupplemented control. The greatest increase in viability and viable cell density was seen in cells grown in DMEM/F12 supplemented with between 10-50 μM spermine. Viable cell density increased by up to 49% and cell viability was increased up to 20% in the spermine-supplemented media, compared to unsupplemented control media.

### Example 2

The antibody-expressing CHO cell line described in Example 1 was also grown in serum-free, soy hydrolysate containing media (VM-Soy, described in US Patent Application No. US2006-0115901), supplemented with spermine. VM-Soy basal media contains 0.98 μM (0.1620 mg/l) putrescine as a component of its formulation. The media was supplemented with spermine tetrahydrochloride (Sigma-Aldrich) to a concentration of 10-200 μM, each concentration was run in duplicate. Cells were seeded at 2.5×10⁵ cells/ml and maintained for six days at 36° C. in 5% CO₂. Viable cell density and cell viability were measured as described above. Once again cell viability and viable cell density were higher in those cultures supplemented with spermine compared to the unsupplemented control media (see FIGS. 2a and 2b). Viable cell density increased 8-23% and cell viability increased 22-29% compared to the unsupplemented control. The greatest increases in viable cell density and cell viability, 23% and 29% respectively, were seen with cells grown in media supplemented with 50 μM spermine.

Antibody titer was measured by immunoturbidietric analysis using a Poly-Chem® analyzer and High Sensitivity IgG reagents (Polymedco, Cortlandt Manor, N.Y.) according to the manufacturer's instructions.

Antibody titer was greater in those cells cultured in the spermine-supplemented media, increasing 5-9% compared to the untreated control. Again, the greatest increase in antibody titer, 9%, was seen with those cells grown in media supplemented with 50 μM spermine.

### Example 3

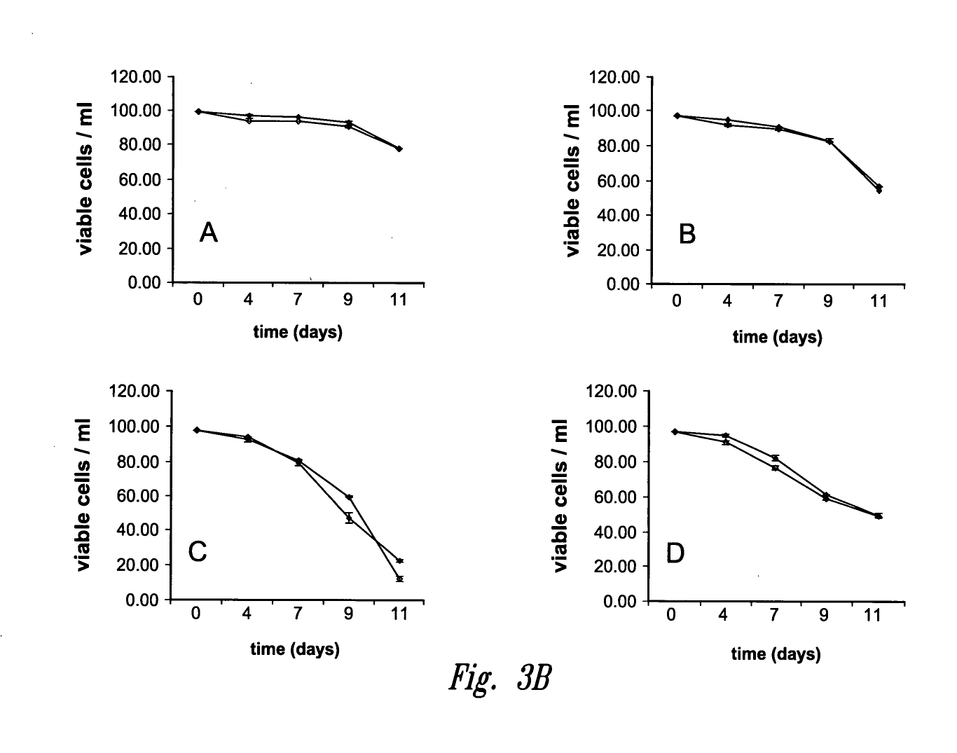

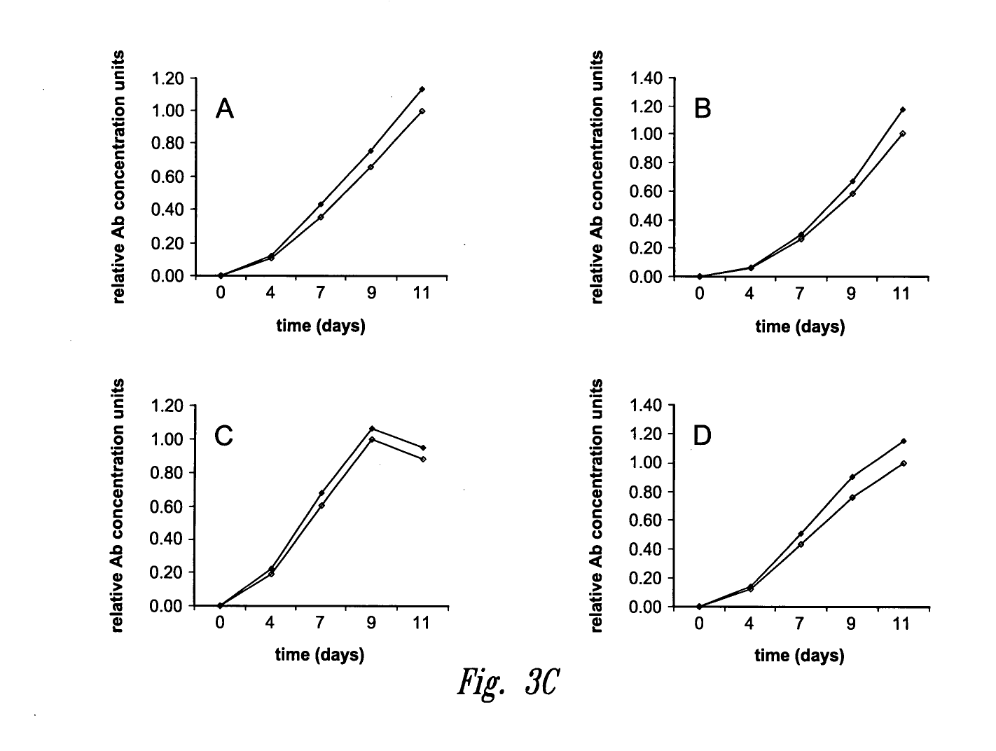

To test the effect of spermine on cells in fed-batch production culture, four different recombinant monoclonal IgG2 antibody-producing CHO cell lines (each expressing a different IgG2 monoclonal antibody) were seeded at 5.0×10⁵ cells/ml in serum-free, hydrolysate-free batch medium. The medium was an enriched formulation of DMEM/F12 medium (ingredients supplied by SAFC, Lenexa, Kans.). The medium contained putrescine at a concentration of 0.014 mM. In this experiment, cells were cultured in medium with or without 10 μM spermine tetrahydrochloride (Sigma-Aldrich). Cells were maintained in suspension culture for 11 days at 36° C., 5% CO₂. Cultures were fed an enriched feed medium on the fourth, seventh, and ninth days of culture. The feed volume was equal to nine percent of the initial batch culture volume. The feed medium contained putrescine but not spermine and delivered 0.0025 mM putrescine as the final concentration in the cultures as a result of each feed. Glucose was also fed on the fourth, seventh, and ninth days of culture in order to prevent depletion. Viable cell density and cell viability were measured as described above. In the fed-batch culture method, viable cell density was greater in cultures that were supplemented with spermine (FIG. 3a). Cell viability was largely unaffected (FIG. 3b).

The titer of the antibodies produced in cell culture was determined using POROS® (Applied Biosystems) Protein A chromotagraphy HPLC chromotagraphy. For each cell line tested, antibody titer was greater in those cells cultured in the spermine-supplemented media, increasing 7-17% compared to the untreated controls (FIG. 3c).

### Example 4

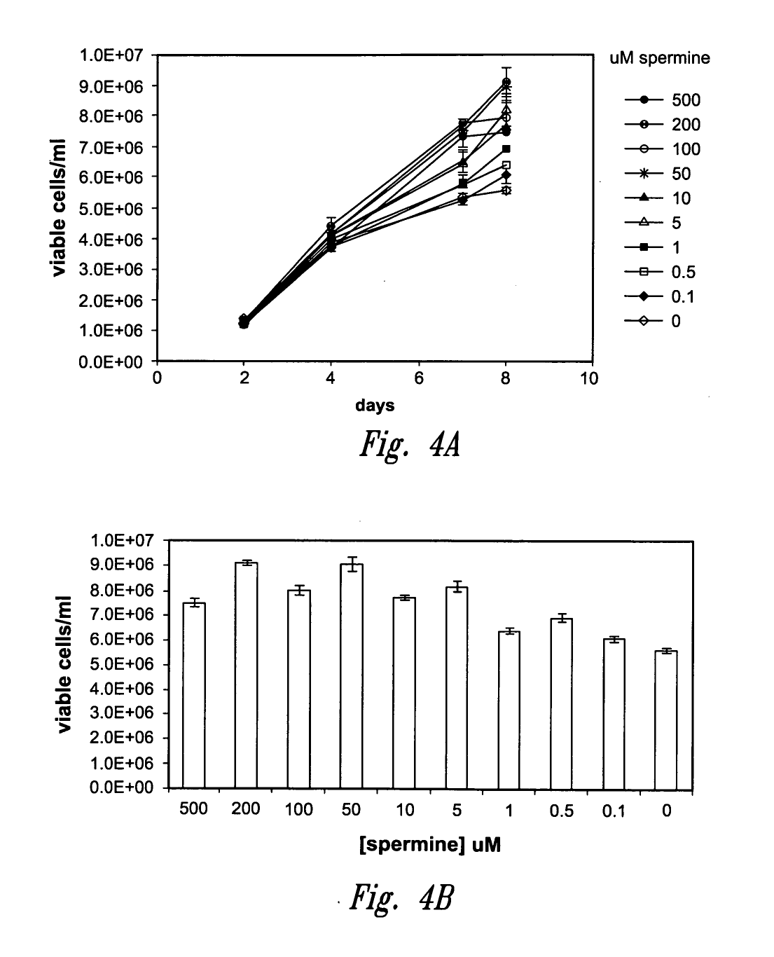

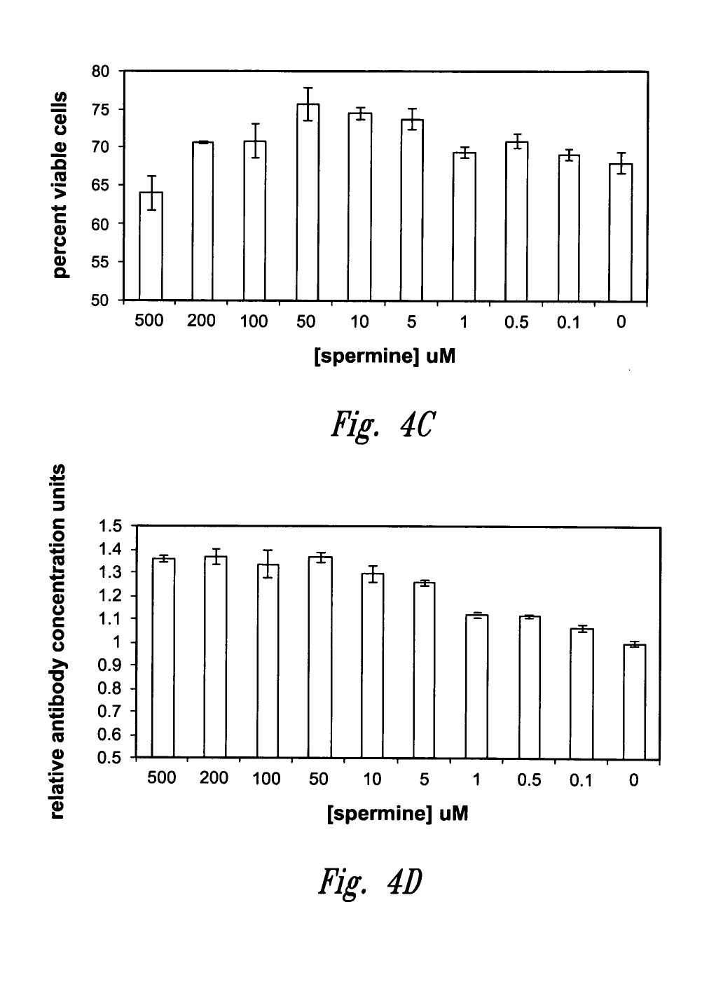

To test the effect of spermine concentration on cells in fed-batch production culture, a CHO cell line producing a recombinant IgG1 monoclonal antibody was seeded at 5.0×10⁵ cells/ml in serum-free, hydrolysate-free medium. The medium was an enriched formulation of DMEM/F12 that was less concentrated than the media used in Example 3. The medium already contained putrescine at 0.014 mM. In this example cells were cultured in batch medium that was supplemented 0.1-500 μM spermine tetrahydrochloride (Sigma-Aldrich). Cells were maintained in suspension culture for 8 days at 36° C., 5% CO₂. Cultures were fed a rich medium on the fourth day of culture. The feed medium contained many of the same components as the batch medium, but at a higher concentration and did not contain spermine. The feed volume was equal to seven percent of the initial batch culture volume. The feed medium contained putrescine and delivered 0.0019 mM putrescine to the cultures as a result of each feed. Glucose was also fed on the fourth day of culture in order to prevent depletion. Viable cell density and cell viability were measured as described above. In the fed-batch culture method, viable cell density was greater in cultures that were supplemented with spermine (FIGS. 4a and 4b). Ending cell viability was increased in cultures dosed with 5-50 μM spermine (FIG. 4c).

Antibody titer was measured by immunoturbidimetric analysis using a Poly-Chem® analyzer and High Sensitivity IgG reagents (Polymedco, Cortlandt Manor, N.Y.) according to the manufacturer's instructions. Antibody titer was greater in those cells cultured in the spermine-supplemented media, increasing 7-17% compared to the untreated controls (FIG. 4d). The spermine had a dose-dependent response on titer and was effective at increasing titer at all the tested concentrations.

Although putrescine is sometimes included as a component of cell culture media, increasing the putrescine concentration from micromolar to millimolar amounts enhanced cell culture performance. Addition of spermidine or spermine to cell culture media enhanced performance of cells in culture with respect to growth, viability, and/or production of recombinant antibodies, spermine having the greatest impact on these parameters.

What is claimed is:

1. A method comprising culturing a CHO cell line in serum free cell culture medium, wherein the CHO cell line expresses a protein of interest and the serum free cell culture medium comprises spermine at a concentration of at least about 0.10 μM, whereby cell viability, viable cell density and expression of said protein of interest are improved relative to CHO cells grown without spermine.

2. The method according to claim 1, wherein the concentration of spermine is at least about 0.10 μM to at least about 500 μM.

3. The method according to claim 1, wherein the concentration of spermine is at least about 10 μM to at least about 200 μM.

4. The method according to claim 1, wherein the concentration of spermine is at least about 50 μM.

5. The method according to claim 1, wherein said protein is an antibody selected from the group consisting of:
a. a human antibody;
b. a humanized antibody;
c. a chimeric antibody;
d. a monoclonal antibody;
e. a multispecific antibody;
g. an antigen binding antibody fragment;
h. a single chain antibody;
i. a diabody;
j. a triabody;
k. a tetrabody;
l. a Fab fragment;
m. a F(ab′)₂ fragment, and
n. an IgG antibody.

6. The method according to claim 5, wherein said antibody is a monoclonal antibody.

7. The method of claim 1, wherein the serum free culture media is a peptone free media comprising spermine at a concentration of at least about 0.10 μM.

8. A cell culture comprising a CHO cell line in serum free cell culture medium, wherein the CHO cell line expresses a protein of interest and the serum free cell culture medium comprises spermine at a concentration of at least about 0.10 μM.

9. A cell culture according to claim 8, wherein said cell culture medium comprises spermine at a concentration from at least about 0.10 μM to at least about 500 μM.

10. A cell culture according to claim 8, wherein said cell culture medium comprises spermine at a concentration of at least about 10 μM to at least about 200 μM.

11. A cell culture according to claim 8, wherein said cell culture medium comprises spermine at a concentration of at least about 50 μM.

12. A cell culture according to claim 8, wherein said protein is an antibody selected from the group consisting of:
a. a human antibody;
b. a humanized antibody;
c. a chimeric antibody;
d. a monoclonal antibody;
e. a polyclonal antibody;
f. a recombinant antibody;
g. an antigen binding antibody fragment;
h. a single chain antibody;
i. a diabody;
j. a triabody;
k. a tetrabody;
l. a Fab fragment;
m. a F(ab′)₂ fragment; and
n. an IgG antibody.

13. A cell culture according to claim 12, wherein said antibody is a monoclonal antibody.

14. A cell culture according to claim 8, wherein the serum free culture media is peptone free media comprising spermine at a concentration of at least about 0.10 μM.

15. A cell culture according to claim 8, wherein said cell culture is supplemented by the use of a concentrated feed medium wherein said feed medium comprises spermine such that the concentration of spermine, when added to the culture, is of at least about 0.10 μM.

16. A method comprising culturing a CHO cell line in serum free cell culture medium, wherein the CHO cell line expresses a protein of interest and the serum free cell culture medium comprises spermidine at a concentration of at least about 0.10 μM or putrescine at a concentration of 100 μM, whereby cell viability and viable cell density are improved relative to CHO cells grown without spermidine at a concentration of at least about 0.10 μM or CHO cells grown without putrescine at a concentration of at least about 100 μM.
